SEQUENCE LISTING (5 sequences)

SEQ ID NO: 1 - protein, 65 aa, Artificial Sequence
  Description of Artificial Sequence Synthetic polypeptide
DAPANPCCDAATCKLTTGSQCADGLCCDQCKFMKEGTVCRRARGDDLDDYCNGISAGCPRNPFHA

SEQ ID NO: 2 - protein, 66 aa, Artificial Sequence
  Description of Artificial Sequence Synthetic polypeptide
GDAPANPCCDAATCKLTTGSQCADGLCCDQCKFMKEGTVCRRARGDDLDDYCNGISAGCPRNPFHA

SEQ ID NO: 3 - protein, 69 aa, Artificial Sequence
  Description of Artificial Sequence Synthetic polypeptide
GDAPANPCCDAATCKLTTGSQCADGLCCDQCKFMKEGTVCRRARGDDLDDYCNGISAGCPRNPHKGPAT

SEQ ID NO: 4 - protein, 49 aa, Artificial Sequence
  Description of Artificial Sequence Synthetic polypeptide
ECESGPCCRNCKFLKEGTICKRARGDDMDDYCNGKTCDCPRNPHKGPAT

SEQ ID NO: 5 - DNA, 207 bp, Artificial Sequence
  Description of Artificial Sequence Synthetic polynucleotide
ggagacgctcctgcaaatccgtgctgcgatgctgcaacatgtaaactgacaacagggtcacagtgtgcagatggactgtgttgtgaccagtgcaaatttatgaaagaaggaacagtatgccggagagcaaggggtgatgacctggatgattactgcaatggcatatctgctggctgtcccagaaatccccacaagggtccagctact

(19) United States
(12) Patent Application Publication
(10) Pub. No.: US 2018/0353627 A1
(43) Pub. Date: Dec. 13, 2018

(54) RADIOLABELED DISINTEGRINS AS BRACHYTHERAPY AGENTS

(71) Applicant: UNIVERSITY OF SOUTHERN CALIFORNIA, Los Angeles, CA (US)

(72) Inventors: Thomas C. Chen, Los Angeles, CA (US); Francis S. Markland, Los Angeles, CA (US); Radu O. Minea, Los Angeles, CA (US); Stephen D. Swenson, Los Angeles, CA (US)

(21) Appl. No.: 15/775,743

(22) PCT Filed: Nov. 11, 2016
(86) PCT No.: PCT/US2016/061603

§ 371 Date: May 11, 2018

Related U.S. Application Data
(60) Provisional application No. 62/255,324, filed on Nov. 13, 2015.

(51) Int. Cl. A61K 51/08; A61K 38/17; A61P 35/00

(52) U.S. Cl. CPC A61K 51/08; A61P 35/00; A61K 38/1703

(57) ABSTRACT

A method for suppressing or inhibiting the growth of a cancer cell or tumor cell that expresses one or more integrins on its cell surface in a subject in need thereof is disclosed, herein, the method comprising administering to the subject an effective amount of contortrostatin (CN) and/or vicrostatin (VCN) or an equivalent of each thereof and wherein the CN and/or VCN or the equivalent of each thereof is conjugated to a therapeutic radioisotope.

### CROSS-REFERENCE TO RELATED APPLICATION

This application claims priority under 35 U.S.C. § 119(e) to U.S. provisional application Ser. No. 62/255,324, filed Nov. 13, 2015, the entire content of which is incorporated by reference into the present disclosure.

### STATEMENT OF GOVERNMENT SUPPORT

This invention was made with government support under Contract No. 1R41CA165626-01A1, awarded by the National Institutes of Health (NIH). The government has certain rights in the invention.

### SEQUENCE LISTING

The instant application contains a Sequence Listing which has been submitted electronically in ASCII format and is hereby incorporated by reference in its entirety. Said ASCII copy created on Nov. 3, 2016, is named 064189-7251_SL.txt and is 3,482 bytes in size.

### BACKGROUND

Despite advances in surgery, chemotherapy, and radiation therapy, the average life span of a patient with glioblastoma multiforme (GBM) is about 15-months from time of diagnosis. Thus, a need exists for effective treatment of GBM and other solid tumors.

### SUMMARY

Integrins are heterodimeric receptors found on the surface of GBM cells and angiogenic vasculature. In the activated state, they facilitate invasion into adjacent tissue. Normal brain tissue does not display activated integrins. Therapy for GBM based on integrin antagonism is disclosed herein as it targets both the neovasculature and the tumor itself.

Disintegrins bind with high affinity to a subset of activated human integrins involved in GBM and angiogenic endothelial cell invasion leading to inhibition of tumor dissemination. The disclosed methods use a novel brachytherapeutic agent that comprise, or alternatively consist essentially of, or yet further consist of, peptides known as disintegrins. In one aspect, the disintegrin comprise, or alternatively consists essentially of, or yet further consists of vicrostatin (VCN). VCN is a recombinant single-chain peptide with all 10 cysteine residues involved in disulfide bond formation.

The advantages of VCN as GBM therapeutic agent include, but are not limited to: (i) the ability of VCN to disrupt the locomotor apparatus of the cell (actin cytoskeleton) and dramatically inhibit invasiveness of both GBM cells and angiogenic vasculature; (ii) minimal off-target effects due to lack of activated integrin expression in normal brain tissue; (iii) stable peptide enabling better penetration through the blood-tumor barrier; (iv) an exclusive recombinant production method that is robust, low cost and easily scalable; and (v) stability of VCN to iodination with only a single tyrosine residue (Y51 in the amino acid sequence) iodinated.

Thus in one aspect, this disclosure provides a method for suppressing or inhibiting the growth of a cancer cell or tumor cell that expresses one or more integrins on its cell surface, comprising, or alternatively consisting essentially of, or yet further consisting of, contacting the cell with an effective amount of contortrostatin (CN) and/or vicrostatin (VCN) or an equivalent of each thereof, and wherein the CN and/or VCN or the equivalent of each thereof is conjugated to one or more therapeutic radioisotopes. The contacting can be in vitro or in vivo. Also provide is a method for suppressing or inhibiting the growth of a cancer cell or tumor cell that expresses one or more integrins on its cell surface in a subject in need thereof, comprising, or alternatively consisting essentially of, or yet further consisting of, administering to the subject an effective amount of contortrostatin (CN) and/or vicrostatin (VCN) or an equivalent of each thereof, and wherein the CN and/or VCN or the equivalent of each thereof is conjugated to one or more therapeutic radioisotopes.

In one aspect of the above methods the one or more integrins is selected from the group of αvβ3, αvβ5, or α5β1 or equivalents of each thereof and non-limiting examples of cells include a cancer cell or tumor cell is selected from the group of a breast cancer cell, an ovarian cancer cell, a pancreatic cancer cell, a renal cell carcinoma, a glioblastoma cell or metastatic foci of each thereof.

In a further aspect of the above methods, the CN and VCN or the equivalent of each thereof are conjugated to the same or different therapeutic radioisotopes.

The methods are useful for a cell of any appropriate species, e.g., a mammalian cell such as a human cell. When administered to a subject, the subject can be any appropriate subject, such as a mammal or a human patient. While any appropriate method of administration can be utilized by the treating physician or veterinarian, in one aspect the compositions are administered by intravenous injection near the cancer or tumor and/or injection into the tumor.

Also provided are pharmaceutical compositions comprising, or alternatively consisting essentially of, or yet further consisting of, an effective amount of contortrostatin (CN) and/or vicrostatin (VCN) or an equivalent of each thereof, and wherein the CN and/or VCN or the equivalent of each thereof is conjugated to one or more therapeutic radioisotopes, and a pharmaceutically acceptable carrier. In a further aspect, provided herein is a dosage formulation comprising, or alternatively consisting essentially of, or yet further consisting of, an effective amount of contortrostatin (CN) and/or vicrostatin (VCN) or an equivalent of each thereof, and wherein the CN and/or VCN or the equivalent of each thereof is conjugated to one or more therapeutic radioisotopes, and a pharmaceutically acceptable carrier.

Yet further provided is a kit comprising, or alternatively consisting essentially of, or yet further consisting of, a pharmaceutical composition comprising an effective amount of contortrostatin (CN) and/or vicrostatin (VCN) or an equivalent of each thereof, and wherein the CN and/or VCN or the equivalent of each thereof is conjugated to one or more therapeutic radioisotopes, and instructions for use.

In one particular aspect, it is noted that since VCN is able to target integrins expressed on the luminal surface of GBM endothelium, the compositions as described herein, e.g., 131I-VCN, can be administered either intratumorally (IT) or intravenously (IV). Brachytherapy for malignant gliomas has been traditionally performed via invasive radioactive probes placed intracranially into the glioma. The use of a novel non-invasive IV delivery modality with uniform distribution throughout the tumor enables the therapeutic compositions (e.g. 131I-VCN) to selectively target integrins overexpressed on the angiogenic tumor endothelial cells and glioma cells.

Thus, in view of the above, provided herein are methods for suppressing or inhibiting the growth of a cancer cell or a tumor cell that expresses an integrin on its cell surface, by contacting the cell and/or administering to the cell or a subject having the tumor or the cancer cell an effective amount of controtorstatin (CN) and/or vicrostatin (VCN) or an equivalent of each thereof, and wherein the CN and/or VCN or the equivalent of each thereof, is conjugated to one or more therapeutic radioisotopes. The radiolabeled CN and/or VCN can be administered as a composition or formulation as described above or contacted with the cell in vitro or in vivo. Non-limiting examples of the therapeutic radioisotope are iridium (Ir)-192, palladium (Pd)-103, iodine (I)-125, iodine (I)-124, iodine (I)-131, and iodine (I)-123. In another aspect, the CN or VCN or the equivalent of each thereof is conjugated to a plurality of the same or different therapeutic radioisotopes.

Currently, no form of targeted intravenous brachytherapy is available in the clinic. All forms of brachytherapy that are currently available are based on local or intratumor administration of radioactivity (in the form of a radioactive pellet etc.). This disclosure provides methods that are different from current and prior art methods and provide unexpected advantages.

One advantage of the current methods is the targeted administration of beta-emitting radionuclides (in one aspect, I-131) into hard to reach tumors or metastatic foci. This cannot be accomplished from the outside the tumor or foci as beta radiation is non-penetrating and only travels for very short path lengths. By delivering beta-emitting radionuclides intravenously in a targeted manner, this ensures a good tumor localization for this type of radioactivity via tumor integrin binding. The integrin set that is targeted by CN/VCN is only expressed by solid tumors (breast, ovarian, pancreatic, renal cell carcinoma or glioblastoma etc.) and their metastatic foci, but not by any normal tissues. Thus, the targeting of DNA-shattering beta-radiation specifically into solid tumors and their metastatic foci guarantees localized tumor destruction (i.e., damage is limited to very short path lengths), the sparing of the normal tissues, and, high beta delivery over short half-life (i.e., 8 days for I-131).

### BRIEF DESCRIPTION OF THE FIGURES

FIG. 1: U87 Mice Luminescent Images on Day 21 of Treatment. Note scale is different for each U87 study: ¹³¹I scale is 10⁸ with a max of 3.85×10⁸ photons/sec/cm² for the U87 ¹³¹I, while the U87¹³¹I-VCN scale is 10⁵ with a maximum of 1.20×10⁵ photons/sec/cm².

FIGS. 2A & 2B: Survival of tumor bearing mice (panel A U87 panel B U251). Animals were either left untreated (control) treated with radioiodine alone (¹³¹I) or radioiodinated VCN (¹³¹I-VCN). The animals were followed throughout the study and either died or were euthanized when their body condition score was <2 (meaning death imminent).

FIG. 3 depicts amino acid sequences of this disclosure. Mass spectrometry and crystallographic data confirmed that CN (SEQ ID. NO. 1) is a homodimer with the 2 chains oriented in an antiparallel fashion and held together by 2 inter-chain disulfide bonds. Underlines show the sequence of echistatin that was incorporated into VCN to improve binding affinity to integrin a5b1 by approximately 13-fold. VCN has an amino-terminal Gly, and six amino acids at the COOH-end from echistatin, the remaining 62 amino acids are identical to those of CN for a total of 69 amino acids. Recombinant CN is SEQ ID NO. 2. VCN is SEQ ID NO. 3. Echistatin is SEQ ID NO. 4.

### BRIEF DESCRIPTION OF THE SEQUENCES

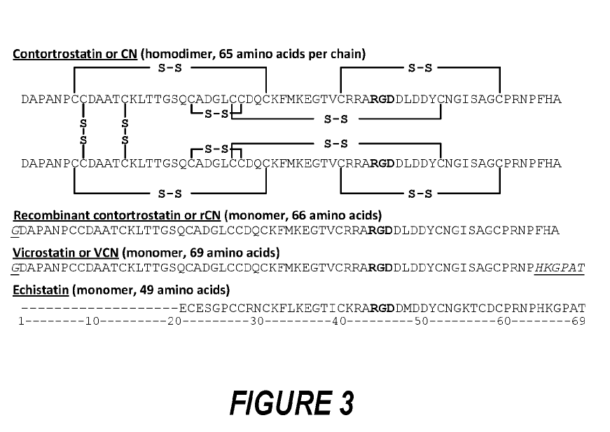

CN is SEQ ID. NO. 1, which as noted above, is a homodimer with the 2 chains oriented in an antiparallel fashion and held together by 2 inter-chain disulfide bonds, see FIG. 3.

Recombinant CN is SEQ ID NO. 2.

The amino acid sequence of VCN is SEQ ID NO. 3.
GDAPANPCCDAATCKLTTGSQCADGLCCDQCKFMKEGTVCRRARGDDLDD
YCNGISAGCPRNPHKGPAT

Echistatin is SEQ ID NO. 4.

Vicrostatin (VCN) DNA sequence is (SEQ ID NO.: 5), depicted below.

cDNA sequence
GGAGACGCTCCTGCAAATCCGTGCTGCGATGCTGCAACATGTAAACTGAC
AACAGGGTCACAGTGTGCAGATGGACTGTGTTGTGACCAGTGCAAATTTA
TGAAAGAAGGAACAGTATGCCGGAGAGCAAGGGGTGATGACCTGGATGAT
TACTGCAATGGCATATCTGCTGGCTGTCCCAGAAATCCCCACAAGGGTCC
AGCTACT

### DETAILED DESCRIPTION OF THE DISCLOSURE

Throughout this application, the text refers to various embodiments of the present compositions and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

Also throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this invention pertains.

### Definitions

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients. Embodiments defined by each of these transition terms are within the scope of this invention.

All numerical designations, e.g., pH, temperature, time, concentration, and molecular weight, including ranges, are approximations which are varied (+) or (−) by increments of 0.1 or 1.0 as is appropriate. It is to be understood, although not always explicitly stated that all numerical designations are preceded by the term “about” which includes a standard deviation of about 15%, or alternatively about 10% or alternatively about 5%. It also is to be understood, although not always explicitly stated, that the reagents described herein are merely exemplary and that equivalents of such are known in the art.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Integrins are heterodimers composed of alpha and beta subunits that are non-covalently associated. Interactions between integrins and extracellular matrix (ECM) proteins have been shown to be mediated via an Arg-Gly-Asp (RGD) sequence present in the matrix proteins. Both the alpha and beta subunits of the integrin are required for ECM protein binding. A wide variety of integrins are found on the surface of solid tumor cells such as GBM cells, melanoma cells, breast cancer cells, ovarian cancer cells, cervical cancer cells, prostate cancer cells, pancreatic cancer cells, non-small-cell lung carcinoma cells, colon cancer cells, renal cell carcinoma cells, colon cancer cells, and metastatic foci of each thereof. These integrins include but are not limited to a6β4, a6β1, αvβ3, αvβ5, a2β1, a3β1, a5β1, a6β4, a4β1 and αvβ6. Integrins may contribute to migration, proliferation and survival in tumor cells. Integrin expression can vary between normal and tumor tissue.

A well-known inhibitor of the integrin-ECM interaction is a disintegrin which represents a family of proteins that include those from venom of snakes of the Crotalidae and Viperidae. Disintegrin families have been found to inhibit glycoprotein (GP) IIb/IIIa mediated platelet aggregation. Disintegrins are disulfide rich and many contain an RGD (Arg-Gly-Asp) sequence that has been implicated in the inhibition of integrin-mediated interactions.

Contortrostatin (CN) is the disintegrin isolated from Agkistrodon contortrix contortrix (southern copperhead) venom. CN displays the classical RGD motif in its integrin-binding loop. Unlike other monomeric disintegrins, CN is a homodimer with a molecular mass (Mr) of 13,505 for the intact molecule and 6,750 for the reduced chains as shown by mass spectrometry. Receptors of CN identified so far include integrins αIIbβ3, αvβ3, αvβ5, and α5β1. Interactions between CN and integrins are all RGD-dependent. As an anti-cancer agent, CN has shown to be a powerful anti-angiogenic and anti-metastatic molecule in in vitro cell-based functional assays and in vivo animal models. CN also has the ability to directly engage tumor cells and suppress their growth in a cytostatic manner. The antitumoral activity of CN is based on its high affinity interaction with integrins α5β1, αvβ3 and αvβ5 on both cancer cells and newly growing vascular endothelial cells. This diverse mechanism of action provides CN with a distinct advantage over many antiangiogenic agents that only block a single angiogenic pathway and/or do not directly target tumor cells.

CN is produced in the snake venom gland as a multidomain precursor of 2027 bp having a 1449 bp open reading frame (encoding proprotein, metalloproteinase and disintegrin domains), which is proteolytically processed, possibly autocatalytically, to generate mature CN. The CN disintegrin domain encodes 65 amino acids with a molecular weight equal to that of the CN subunit. The CN full-length precursor mRNA sequence can be accessed in the GeneBank database using accession number: AF212305. The nucleotide sequence encoding the 65 amino acid disintegrin domain of CN represents the segment from 1339 to 1533 in the mRNA.

Structurally, CN is a cysteine-rich protein (10 cysteines per monomer) that displays no secondary structure and, like other disintegrins, has a complex folding pattern that relies on multiple disulfide bonds (four intrachain and two interchain disulfide bonds) to stabilize its tertiary structure. By folding in a compact structure locked by multiple disulfide bonds, CN, like many other venom proteins, has a survival advantage, being less susceptible to a proteolytic attack and better equipped to survive in the harsher extracellular microenvironment. Its highly cross-linked structure and unique biological activity are barriers to producing biologically functional CN (or other disintegrin domain protein) using a recombinant expression system. A further difficulty is that the CN disintegrin domain of the multidomain precursor, from which dimeric CN is derived, displays no secondary structure, a feature that is known to facilitate the proper folding in most nascent proteins. The crystal structure of native CN has not been elucidated. CN's folding pattern is presumably as complex as other viperid dimeric disintegrins that have been studied.

U.S. Pat. No. 7,754,850 describes vicrostatin (VCN), a recombinant fusion protein wherein the last three amino acids of the carboxy terminus of CN are swapped with the C-terminal tail of echistatin (HGKPAT) The amino acid sequence of VCN is disclosed in SEQ ID NO. 3. The N-terminal Glycine residue in SEQ ID NO. 3 results from a post expression processing of an N-terminal thioredoxin fusion having a TEV protease linker site. The N-terminus of VCN may lack the Glycine or may have some other amino acid(s) without impacting the activity of the molecule. The C-terminus has six amino acids from echistatin (SEQ ID NO. 4). The remaining 62 amino acids in the sequence are identical to those of CN.

A “subject” of diagnosis or treatment is a cell or an animal such as a mammal, or a human. Non-human animals subject to diagnosis or treatment and are those subject to GBM or animal models, for example, simians, murines, such as, rats, mice, chinchilla, canine, such as dogs, leporids, such as rabbits, felines, livestock, sport animals, and pets.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “isolated” or “recombinant” as used herein with respect to nucleic acids, such as DNA or RNA, refers to molecules separated from other DNAs or RNAs, respectively that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

It is to be inferred without explicit recitation and unless otherwise intended, that when the present invention relates to a polypeptide, protein, polynucleotide or antibody, an equivalent or a biologically equivalent of such is intended within the scope of this invention. As used herein, the term “biological equivalent thereof” is intended to be synonymous with “equivalent thereof” when referring to a reference protein, antibody, fragment, polypeptide or nucleic acid, intends those having minimal homology while still maintaining desired structure or functionality. Unless specifically recited herein, it is contemplated that any polynucleotide, polypeptide or protein mentioned herein also includes equivalents thereof. In one aspect, an equivalent polynucleotide is one that hybridizes under stringent conditions to the polynucleotide or complement of the polynucleotide as described herein for use in the described methods.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity were determined by incorporating them into clustalW (available at the web address://align.genome.jp/, last accessed on Mar. 7, 2011.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

As used herein, the term “encode” refers to any process whereby the information in a polymeric macromolecule or sequence string is used to direct the production of a second molecule or sequence string that is different from the first molecule or sequence string. As used herein, the term is used broadly, and can have a variety of applications. In one aspect, the term “encode” describes the process of semi-conservative DNA replication, where one strand of a double-stranded DNA molecule is used as a template to encode a newly synthesized complementary sister strand by a DNA-dependent DNA polymerase. In another aspect, the term “encode” refers to any process whereby the information in one molecule is used to direct the production of a second molecule that has a different chemical nature from the first molecule. For example, a DNA molecule can encode an RNA molecule (e.g., by the process of transcription incorporating a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the radioisotope employed, bioavailability of the compound, the route of administration, the age of the subject or patient and its body weight, general health, sex, the diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of diseases. Thus, where a composition is found to demonstrate in vitro activity, for example as noted in the examples provided below, one can extrapolate to an effective dosage for administration in vivo. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response for treating a cancer cell or tumor as described herein.

As used herein, “treating” or “treatment” of a disease in a patient refers to (1) preventing the symptoms or disease from occurring in an animal that is predisposed or does not yet display symptoms of the disease; (2) inhibiting the disease or arresting its development; (3) ameliorating or causing regression of the disease or the symptoms of the disease; or (4) reducing the metastasis of the tumor or cancer. As understood in the art, “treatment” is an approach for obtaining beneficial or desired results, including clinical results. For the purposes of this invention, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like.

A “control” is an alternative subject or sample used in an experiment for comparison purpose. A control can be “positive” or “negative”. For example, where the purpose of the experiment is to determine a correlation of the efficacy of a composition of the invention for the treatment for a particular type of disease or cancer, it is generally preferable to use a positive control (a compound or composition known to exhibit the desired therapeutic effect) and a negative control (a subject or a sample that does not receive the therapy or receives a placebo).

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition ofa cancer cell, as used herein, includes not only a primary cancer cell, but also any cell derived from a cancer cell ancestor. This includes metastasized cancer cells, metastatic foci, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

A neoplasm is an abnormal mass or colony of cells produced by a relatively autonomous new growth of tissue. Most neoplasms arise from the clonal expansion of a single cell that has undergone neoplastic transformation. The transformation of a normal to a neoplastic cell can be caused by a chemical, physical, or biological agent (or event) that directly and irreversibly alters the cell genome. Neoplastic cells are characterized by the loss of some specialized functions and the acquisition of new biological properties, foremost, the property of relatively autonomous (uncontrolled) growth. Neoplastic cells pass on their heritable biological characteristics to progeny cells.

The past, present, and future predicted biological behavior, or clinical course, of a neoplasm is further classified as benign or malignant, a distinction of great importance in diagnosis, treatment, and prognosis. A malignant neoplasm manifests a greater degree of autonomy, is capable of invasion and metastatic spread, may be resistant to treatment, and may cause death. A benign neoplasm has a lesser degree of autonomy, is usually not invasive, does not metastasize, and generally produces no great harm if treated adequately.

Cancer is a generic term for malignant neoplasms. Anaplasia is a characteristic property of cancer cells and denotes a lack of normal structural and functional characteristics (undifferentiation).

A tumor is literally a swelling of any type, such as an inflammatory or other swelling, but modem usage generally denotes a neoplasm. The suffix “-oma” means tumor and usually denotes a benign neoplasm, as in fibroma, lipoma, and so forth, but sometimes implies a malignant neoplasm, as with so-called melanoma, hepatoma, and seminoma, or even a non-neoplastic lesion, such as a hematoma, granuloma, or hamartoma. The suffix “-blastoma” denotes a neoplasm of embryonic cells, such as neuroblastoma of the adrenal or retinoblastoma of the eye.

Histogenesis is the origin of a tissue and is a method of classifying neoplasms on the basis of the tissue cell of origin. Adenomas are benign neoplasms of glandular epithelium. Carcinomas are malignant tumors of epithelium. Sarcomas are malignant tumors of mesenchymal tissues. One system to classify neoplasia utilizes biological (clinical) behavior, whether benign or malignant, and the histogenesis, the tissue or cell of origin of the neoplasm as determined by histologic and cytologic examination. Neoplasms may originate in almost any tissue containing cells capable of mitotic division. The histogenetic classification of neoplasms is based upon the tissue (or cell) of origin as determined by histologic and cytologic examination.

“Suppressing” tumor growth indicates a growth state that is curtailed compared to growth without any therapy. Tumor cell growth can be assessed by any means known in the art, including, but not limited to, measuring tumor size, weight, and/or volume, determining whether tumor cells are proliferating using a ³H-thymidine incorporation assay, or counting tumor cells. “Suppressing” tumor cell growth means any or all of the following states: slowing, delaying, inhibiting, and reducing, and “suppressing” tumor growth indicates a growth state that is curtailed when stopping tumor growth, as well as tumor shrinkage, inducing quiescence of a tumor, altering the metabolic activity of a tumor, inhibiting or reducing metastasis in the subject, inhibiting or reducing tumor invasiveness in the subject, and/or improving survival of the subject.

The term “culturing” refers to the in vitro propagation of cells or organisms on or in media of various kinds. It is understood that the descendants of a cell grown in culture may not be completely identical (morphologically, genetically, or phenotypically) to the parent cell. By “expanded” is meant any proliferation or division of cells.

### Description of the Embodiments

### Compositions and Formulations

This disclosure provides an isolated polypeptide of contortrostatin (CN) or an equivalent thereof, conjugated to a therapeutic radioisotope or more than one therapeutic radioisotope. In one aspect, the CN comprises the amino acid sequence of SEQ ID NO.: 1, or an equivalent thereof. In aspect the equivalent of SEQ ID NO.: 1 intends a polynucleotide having at least about 70%, or alternatively at least about 75%, or alternatively at least about 80%, or alternatively at least about 85%, or alternatively at least about 90%, or alternatively at least about 95%, or alternatively at least about 97%, sequence identity to SEQ ID NO. 1. In a further aspect, the equivalent has the substantially the same biological activity as SEQ ID NO.: 1. In a further aspect, the equivalent of SEQ ID NO.: 1 intends a polypeptide encoded by a polynucleotide or the complement thereof, that hybridizes under stringent conditions to the polynucleotide encoding SEQ ID NO: 1, or it complement.

In one embodiment, the therapeutic radioisotope is selected from the group of iridium (Ir)-192, palladium (Pd)-103, iodine (I)-125, iodine (I)-124, iodine (I)-131, and iodine (I)-123. In another aspect, the CN or VCN or the equivalent of each thereof is conjugated to a plurality of therapeutic radioisotopes, that may be the same or different from each other.

This disclosure also provides an isolated polypeptide comprising vicrostatin (VN) or an equivalent thereof, conjugated to a therapeutic radioisotope or more than one therapeutic radioisotope. In one aspect, the wherein the VCN is a polypeptide comprising the amino acid sequence of SEQ ID NO.: 3, or an equivalent thereof. In aspect the equivalent of SEQ ID NO.: 3 intends a polynucleotide having at least about 70%, or alternatively at least about 75%, or alternatively at least about 80%, or alternatively at least about 85%, or alternatively at least about 90%, or alternatively at least about 95%, or alternatively at least about 97%, sequence identity to SEQ ID NO. 3. In a further aspect, the equivalent has the substantially the same biological activity as SEQ ID NO.: 3. In a further aspect, the equivalent of SEQ ID NO.: 3 intends a polypeptide encoded by a polynucleotide or the complement thereof, that hybridizes under stringent conditions to the polynucleotide encoding SEQ ID NO: 3, or it complement.

In one embodiment, the therapeutic radioisotope is selected from the group of iridium (Ir)-192, palladium (Pd)-103, iodine (I)-125, iodine (I)-124, iodine (I)-131, and iodine (I)-123. In another aspect, the CN or VCN or the equivalent of each thereof is conjugated to a plurality of therapeutic radioisotopes, that may be the same or different from each other.

As used herein, the term “conjugated” intends a chemical reaction, that is covalent or non-covalent.

Methods to prepare the isolated polypeptides by recombinant or chemical methods are known in the art. For example, an isolated polynucleotide encoding the polypeptide can be inserted into an appropriate replication vector and inserted into an appropriate host cell, such as a prokaryotic or eukaryotic host cells and replicated by growing the host cell under appropriate conditions and isolated using conventional techniques.

In a further aspect, this disclosure provides a method to prepare the radiolabeled polypeptides of this disclosure by admixing an effective amount of the therapeutic radioisotope(s) and the polypeptide under conditions to conjugate the polypeptide to the therapeutic radioisotope(s), and removing any unconjugated therapeutic radioisotope.

In the one aspect of the above embodiments, an equivalent comprises, or alternatively consists essentially of, or yet further consists of, a polypeptide having at least 70% sequence identity to the CN or VCN. In another aspect, an equivalent comprises a polypeptide encoded by a polynucleotide that hybridizes under stringent conditions to a polynucleotide encoding CN or VCN, respectively or its complement.

In the aspects of the above embodiments, an equivalent of the integrin expressed on the cell surface of a cancer cell or tumor cell comprises, or alternatively consists essentially of, or yet further consists of, a polypeptide or heterodimer of polypeptides having at least 70% sequence identity to αvβ3, αvβ5, or α5β1. In another aspect, an equivalent comprises a polypeptide or heterodimer of polypeptides encoded by a polynucleotide that hybridizes under stringent conditions to a polynucleotide comprising one or more of the integrin subunit genes ITGAV, ITGA5, ITGB1, ITGB3, ITGB5, or homologs thereof, respectively or its complement. Non-limiting examples of species with equivalent orthologs of human integrin subunit genes include sauropsida such as birds and reptiles, and mammals such as primates, rodents, felines, equines, and canines.

In a further aspect, this disclosure provides a plurality of isolated polypeptides as described above, wherein the plurality of the polypeptides in the composition are the same or different from each other.

In another aspect, this disclosure provides a composition comprising a carrier, such as a pharmaceutically acceptable carrier, and isolated polypeptide and/or plurality of polypeptides as described above. The carrier can be selected for intravenous or intratumoral administration to a subject in need thereof.

The compositions can additional contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols.

Administration of the pharmaceutical compositions according to the present invention will typically be via any common route. This includes, but is not limited to parenterally, intratumorally, intravenously, orally, orthotopically, intradermally, intraperitoneally, subcutaneously, intramuscularly, intraperitoneally, intranasally, intrathecally, intraarthricularly, or intraarterially. The active compounds of the present invention can be formulated for parenteral administration, e.g., formulated for injection via the intravenous or intratumoral, routes. The preparation of an aqueous composition that contains the radiolabeled VCN and/or CN will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

In one aspect, this disclosure provides a kit comprising a pharmaceutical composition comprising an effective amount of contortrostatin (CN) and/or vicrostatin (VCN) or an equivalent of each thereof, and wherein the CN and/or VCN or the equivalent of each thereof is conjugated to one or more therapeutic radioisotopes, and instructions for use.

In another aspect, this disclosure provides a dosage formulation comprising an effective amount of contortrostatin (CN) and/or vicrostatin (VCN) or an equivalent of each thereof, and wherein the CN and/or VCN or the equivalent of each thereof is conjugated to one or more therapeutic radioisotopes, and a pharmaceutically acceptable carrier.

### Therapeutic Methods

Provided herein are methods for suppressing or inhibiting the growth of a cancer cell or tumor cell that expresses an integrin on its cell surface, by contacting the cell, in vitro or in vivo, or by administering to a subject having the tumor or the cancer, an effective amount of controtorstatin (CN) and/or vicrostatin (VCN) or an equivalent of each thereof, and wherein the CN and/or VCN or the equivalent of each thereof, is conjugated to a therapeutic radioisotope. The radiolabeled CN and/or VCN can be administered as a composition or formulation as described above. Non-limiting examples of the therapeutic radioisotope are iridium (Ir)-192, palladium (Pd)-103, iodine (I)-125, iodine (I)-124, iodine (I)-131, and iodine (I)-123. In another aspect, the CN or VCN or the equivalent of each thereof is conjugated to different therapeutic radioisotopes or alternatively, is conjugated to the same therapeutic radioisotope. The effective amount is an amount determined to be effective for the subject and the cancer or tumor being treated, taking into consideration the health, weight and other therapies that are or were administered as well as the particular therapeutic radioisotope being administered, as well as the route of administration. These can be empirically determined by the treating physician or veterinarian.

These methods can be a first line, second line, third line, or fourth line therapy when used in combination with additional therapies as known in the art and briefly described herein.

In one aspect, the VCN and/or or the equivalent of each thereof, is administered to the subject by intravenous injection near and/or proximal the cancer or tumor cell and/or by direct injection into the tumor or cell itself.

In another aspect, a plurality of CN or VCN or the equivalent of each thereof is administered to the subject, and each of the CN or VCN or the equivalent of each thereof is conjugated to the same species of radioisotope, e.g., all I-131. In a further aspect, a plurality of CN or VCN or the equivalent of each thereof is administered to the subject, and each of the CN or VCN or the equivalent of each thereof is conjugated to a different therapeutic radioisotope, each polypeptide might be conjugated to I-131 and I-124. Alternatively, one polypeptide might be solely conjugated to a single type of therapeutic radioisotope but co-administered with the same or different polypeptide (e.g., CN-I-131 and CN-I-124 or CN-I-131 and VCN-I-131).

Non-limiting examples of subjects in need thereof are mammals, such as bovines, canines, equines, felines, and human subjects. The radiolabeled CN and/or VCN can be administered as a composition or formulation.

In one aspect, the integrin expressed on the tumor or cancer cell is selected from the group consisting of αvβ3, αvβ5, and α5β1 and homologs, variants, and equivalents thereof. Integrin subunits may be encoded by several genes including, but not limited to, ITGAV, ITGA5, ITGB1, ITGB3, ITGB5, and homologs, variants, and equivalents thereof. A cancer cell or tumor cell that expresses one or more integrins is selected from the group consisting of a breast cancer cell, an ovarian cancer cell, a pancreatic cancer cell, a renal cell carcinoma, a glioblastoma cell and/or metastatic foci of each thereof. In a further aspect, a sample of the tumor is isolated from the subject and assayed for expression of the integrin(s) and the therapy is administered to the subject if the tumor or cancer cell is positive for the expression of the integrin(s). Methods for assaying for integrin expression are known in the art, e.g., immunohistochemical methods.

One of skill in the art can determine if the method has been successful by the treating physician using clinical or subclinical criteria, e.g., reduction in tumor burden, slowing of tumor growth, longer overall survival, longer time to tumor progression, inhibition of metastasis or a reduction in metastasis of the tumor.

In another aspect, this disclosure provides an in vitro method that comprises contacting a cell that expresses an integrin on the cell surface, with an effective amount of the radiolabeled CN and/or radiolabeled VCN, or an equivalent thereof. The in vitro method can be used to assay for effectiveness of the therapy alone or in combination with other therapies, as described herein. The cells can be of any appropriate species, a mammalian cells, a bovine, cell, a canine cell, an equine cell, a feline cell, or a human cell, and the radiolabeled CN and/or VCN can be administered as a composition or formulation as described above. Non-limiting examples of the therapeutic radioisotope are iridium (Ir)-192, palladium (Pd)-103, iodine (I)-125, iodine (I)-124, iodine (I)-131, and iodine (I)-123. In one aspect, the CN or VCN or the equivalent of each thereof is conjugated to different therapeutic radioisotopes or alternatively, is conjugated to the same therapeutic radioisotope. In another aspect, a plurality of CN or VCN or the equivalent of each thereof is contacted with the cell and each of the CN or VCN or the equivalent of each thereof is conjugated to the same therapeutic radioisotope. In a further aspect, a plurality of CN or VCN or the equivalent of each thereof is contacted with the cell and each of the CN or VCN or the equivalent of each thereof is conjugated to a different therapeutic radioisotope.

In aspects of this disclosure, a non-limiting example of VCN is a polypeptide comprising, or alternatively consisting essentially of, or yet further consisting of, the amino acid sequence of SEQ ID NO.: 3, or an equivalent thereof. In an alternative aspect, the equivalent comprises a polypeptide having at least 70% sequence identity, or alternatively at least 80%, or alternatively at least 85%, or alternatively at least 9⁰%, or alternatively at least 95%, or alternatively at least 97% sequence identity, to the VCN. In a yet further aspect, the equivalent comprises a polypeptide encoded by a polynucleotide that hybridizes under stringent conditions to a polynucleotide encoding VCN, respectively or its complement.

In aspects of this disclosure, a non-limiting example of CN a polypeptide comprising, or alternatively consisting essentially of, or yet further consisting of, the amino acid sequence of SEQ ID NO.: 1, or an equivalent thereof. In an alternative aspect, the equivalent comprises a polypeptide having at least 70% sequence identity, or alternatively at least 80%, or alternatively at least 85%, or alternatively at least 90%, or alternatively at least 95%, or alternatively at least 97% sequence identity, to the CN. In a yet further aspect, the equivalent comprises a polypeptide encoded by a polynucleotide that hybridizes under stringent conditions to a polynucleotide encoding CN, respectively or its complement.

### Combination Therapies

The methods as describe above can be combined with other methods known in the art for the treatment of solid tumors, e.g. surgical resection, chemotherapy, radiation therapy, or a combination of these therapies. It can be a first line, second line, third line, or fourth line therapy when used in combination of these additional therapies. When used in combination with a chemotherapy for example, the methods can be administered concurrently or in combination with each other.

### Experimental Methods

Glioblastoma multiforme (GBM) is a devastating brain cancer and the most common primary malignant brain tumor; GBM affects males and females both young and old. The average life span of a patient with GBM is ˜15 months from time of diagnosis. Current treatment consists of surgery, followed by focused beam radiation with oral temozolomide, then temozolomide alone. Patients that fail temozolomide are considered recurrent GBM, and are treated either by repeat surgery, bevacizumab (Avastin), or clinical trials.

Invasiveness of GBM is linked to a family of cell surface proteins called integrins that enable the cancer cells to interact with the surrounding extracellular matrix (ECM) and invade adjacent tissue. GBM cells display a diverse group of these heterodimeric integrin receptors on their surface including αvβ3, αvβ5, and α5β1.

Importantly, these integrins are not expressed in the surrounding normal brain tissue. GBM also has numerous blood vessels (angiogenic vasculature) that deliver oxygen and nutrients to the tumor, facilitate tumor growth, and help bring about GBM dissemination into normal brain tissue. Integrins αvβ3, αvβ5 and α5β1 are also involved in angiogenic vasculature migration and invasion. Thus, therapy against GBM based on integrin antagonism is particularly attractive, and has been shown to exhibit antiangiogenic, antitumor and anti-invasive activities.

Thus this disclosure is related to the development of an integrin-targeted brachytherapy agent using ¹³¹I-vicrostatin (VCN). Recombinant production of VCN is known in the art, e.g., U.S. Publication No. 20060246541, which includes, as an embodiment, expression of a chimeric snake venom disintegrin Vicrostatin (VCN) in the Origami™ B (DE3)/pET32a system. Applicants have shown that VCN, a recombinant disintegrin whose sequence is based on that of contortrostatin (CN), a snake venom disintegrin, can be radioiodinated with full retention of bioactivity (as assessed by inhibition of platelet aggregation). Further, 131I-VCN retained essentially 100% radioiodine during dialysis versus phosphate buffered saline, pH 7.4, for 15 days. Mass spec analysis revealed that the only amino acid modified by radioiodination was Tyr51 in the amino acid sequence of VCN, and the ratio of unmodified/monoiodo/diiodo Tyr51 was 30/50/20.

Using fluorescence polarization to assess binding affinity of I-VCN to αvβ3, Applicants found minimal alteration to the low nM binding affinity (˜7 nM) of native VCN for this integrin. These results validate the use of this agent as a brachytherapy agent with specific targeting to integrins displayed by both GBM cell and angiogenic vasculature. Since VCN does not bind to integrins on mature vessels or on normal non-migrating cells, where integrins are in an unactivated (non-binding) state, ¹³¹I-VCN provides dual activities targeted to GBM—as a potent integrin antagonist blocking invasion, and as an effective brachytherapy agent.

The specific advantages of ¹³¹I-VCN for therapy of GBM include: high integrin binding affinity (low nM IC50s) for integrins αvβ3, αvβ5, and α5β1; ability to disrupt the locomotor apparatus (actin cytoskeleton) of both GBM and angiogenic vascular endothelial cells, which dramatically inhibits their invasive activity; minimal off-target effects due to lack of activated integrin expression on normal brain tissue; stability to iodination without loss of activity and with a single tyrosine residue (Tyr51) iodinated; ability to be delivered either intravenously (i.v.), where the angiogenic vasculature of GBM is targeted in a unique non-invasive modality compared to invasive brachytherapy presently in use, or intratumorally (i.t.), where both GBM and angiogenic endothelial cells will be targeted using delivery via an Alzet pump; and an exclusive recombinant production method that is robust, low cost and easily scalable to provide sufficient VCN to move the unique brachytherapy technology to the clinic.

### Experimental Methods

VCN Preparation:

Vicrostatin (VCN) is the chimeric disintegrin purposefully designed using an engineered recombinant system in E. coli that was obtained by grafting the C-terminal tail of viperid snake venom disintegrin echistatin to the sequence of crotalid disintegrin contortrostatin (CN). (See for example, U.S. Publication no. 20060246541, incorporated herein by reference.)—VCN exhibits identical anticancer properties to venom derived CN.

Toxicology Studies:

The toxicity of single i.v. escalating doses of VCN (3 mg/kg to 75 mg/kg) was evaluated in groups of three rats/dose. Rats were evaluated for signs of physical toxicity or stress and sacrificed after 14 days. There were no adverse effects observed in any of the animals. Animals thrived and gained weight indistinguishable from control. There were no changes in behavior, or in gross or microscopic examination following sacrifice, and no significant differences in hematological parameters. Integrins must be in an activated conformation to bind VCN, and this integrin state is found only in migratory cells such as cancer cells and angiogenic vasculature. Thus, VCN has virtually no toxicity to normal tissue.

Circulatory Half-Life of VCN:

Blood samples were collected following i.v. administration of ¹²⁵I-VCN in tumor-free mice. Gamma counting over time revealed a blood t½ of 6.7 h for ¹²⁵I-VCN.

Animal Tumor Model Studies:

VCN has excellent efficacy in animal models of human cancer with favorable pharmacological attributes and translational potential. Through ligation of integrins αvβ3, αvβ5, and α5β1, VCN targets both endothelial and cancer cells, profoundly disrupting the actin cytoskeleton, ultimately interfering with tumor cell ability to invade and endothelial cell tube formation. Antitumor efficacy of an i.v. liposomal formulation of VCN (LVCN) was evaluated in two breast cancer animal models with different growth characteristics. LVCN was well tolerated and exerted a significant delay in tumor growth and an increase in survival, which can be explained by its potent antiinvasive/antiangiogenic activity.

Iodination of VCN and Stability of I-VCN:

VCN in solution can be directly iodinated, using a modification of the chloramine T method. Briefly, chloramine T is dissolved in PBS and added to a buffered VCN solution containing Na¹²⁵I or Na¹³¹I. Five successive aliquots of 15 μg chloramine T (in 5 μl) are added at 5-min intervals to the reaction mixture. Excess ¹²⁵I or ¹³¹I is quenched through the addition of sodium metabisulfite. Unreacted iodine is removed from the ¹²⁵I- or ¹³¹I-VCN solution by repeated buffer changes and filtration through a 3-kDa cutoff slide-a-lyzer.

The specific activity of iodinated VCN is determined by separation of the material by SDS-PAGE, and counting bands of VCN cut from the gel. One can radioiodinate VCN with full retention of biological activity (assessed by inhibition of platelet aggregation). Further, ¹³¹I-VCN retained essentially 100% radioiodine during dialysis against phosphate buffered saline, pH 7.4, for 15 days. Analysis by mass spectrometry revealed that the only amino acid modified by radioiodination was Tyr51, and the ratio of unmodified/monoiodo/diiodo Tyr51 was 30/50/20. Using fluorescence polarization to assess binding affinity of I-VCN to αvβ3, we found minimal alteration in the low nM binding affinity of native VCN to αvβ3 (˜7 nM) (Table 1). Binding kinetics were calculated from the fluorescence anisotropy data generated by steady state binding of FITC-labeled disintegrins to either purified (αvβ3, αvβ5) or recombinant (α5β1) functional human integrins. The dissociation constants for interactions of either CN or VCN with soluble integrins were determined by Scatchard analysis using a non-linear curve fit.

Iodine incorporation into VCN can be varied by adjusting initial radioiodine levels VCN as well as time of reaction and amount of chloramine T; the stoichiometry of incorporation approaches 1 mol iodine per mol of VCN, to the sole tyrosine residue (Tyr51) in VCN. Importantly, ¹²⁵I-VCN accumulates in the brains of tumor bearing rats (˜2% of the delivered dose), while in control tumor free animals no accumulation (<0.1% of the delivered dose was observed), indicating that an intravenous route of administration is feasible for radioiodinated-VCN. These results validate the use of this agent as a targeted brachytherapeutic agent with specific targeting to integrins displayed by both GBM cell and angiogenic vasculature. Since VCN does not bind to integrins on mature vessels or on normal non-migrating cells, where integrins are in an unactivated (non-binding) state, ¹³¹I-VCN will serve as an effective brachytherapy agent with dual functions—both as a potent integrin antagonist (with IC₅₀s for integrins in the low nM range) and as an effective and targeted brachytherapy agent.

Previous Studies in GBM:

All previous work was done with CN, the precursor of VCN. Applicants previously determined whether CN induces hemorrhage in healthy, tumor-free rats. Hemorrhage was minimal and localized to the injection needle track; there was no difference between PBS-injected brains and brains injected with different concentrations of CN Applicant also examined immune response after CN or lipopolysaccharide (positive control) injection. There was no evidence of an immune response after intracranial injection of 50 μg CN in tumor-bearing (U87 GBM), or in tumor-free rodents at 7 days, or after injecting 150 μg CN after 7, 14 or 21 days.

A Gamma camera was used to determine biodistribution of ¹³¹I-CN in rats after intracranial injection of C6 rat GBM cells, and tumors allowed to grow for 7 days. Two tumor-free rats were also administered ¹³¹I-CN intracranially. Whole-body distribution of radioactivity was imaged at 0 and 24 hours after injection of ¹³¹I-CN.

CN is localized to the brain in all animals at 0 hours. After 24 hours CN had disappeared from the brains of tumor-free rats and was distributed throughout the body. In sharp contrast, in the brains of tumor-bearing animals at 24 hours the vast majority of radioactivity was still retained, predominantly concentrated at the site of tumor injection. This retention was attributable to CN's specific binding to integrin αvβ3, which has been shown previously by us and others to be overexpressed in glioma cells, but not in normal brain tissue. Thus, CN delivered intratumorally binds to the expected integrins in GBM in vivo and accumulates specifically at the site of the tumor. Based on structural and functional similarities between VCN and CN, we anticipate identical findings with VCN for GBM localization. Initial results suggest that ¹³¹I VCN when given i.v. localizes to the intracranial tumor, and not to systemic organs. The ability to specifically accumulate at the site of the tumor is a significant attribute and will prove beneficial for therapeutic application of VCN.

Finally, in an intracranial tumor model, U87 GBM cells were stereotactically implanted into nude mice. Seven days after tumor implantation, Alzet osmotic pumps were implanted in the shoulder, and tygon tubing was used to deliver 40 μg of VCN daily over a period of 2 weeks to the brains of five mice; control U87 GBM bearing mice were similarly administered PBS. After 2 weeks the mice were euthanized and their brains removed. The average tumor size of animals treated with PBS was several times larger than animals treated with VCN. Thus, VCN caused a significant (p<0.001) attenuation of tumor growth. In a survival study, intratumoral delivery of CN via Alzet pump significantly (p<0.008) improved survival of GBM-bearing animals.

Preliminary Brachytherapy Studies

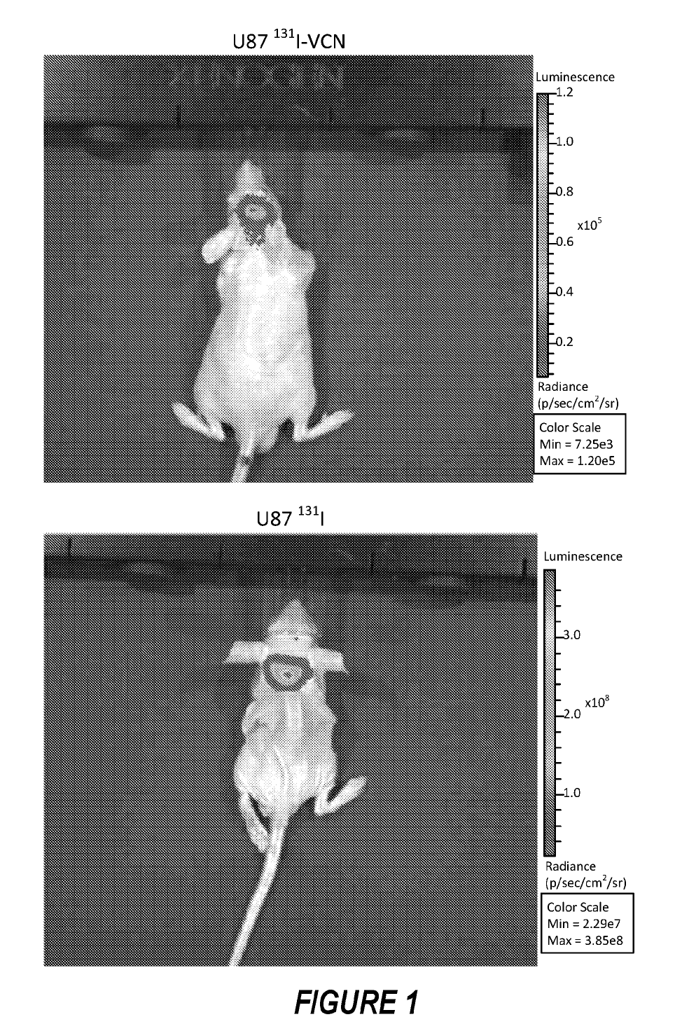

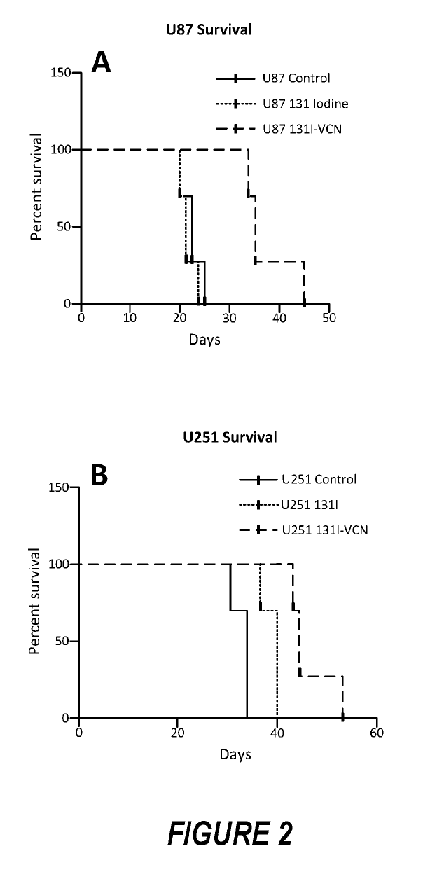

In a number of brachytherapy studies with GBM we were able to show enhanced survival with ¹³¹I-VCN therapy of GBM. In these studies U87 and U251 tumors were implanted in the brains of nude mice (200,000 cells in 5 μl stereotactically implanted 3 mm beneath the skull). The tumors were allowed to grow for 7 days, and at this time the luciferase expressing tumors were imaged via optical imaging procedures (Xenogen). Upon confirmation of tumor growth the mice were randomized into groups (¹³¹I-VCN, ¹³¹I alone, and control). The mice were reimaged on day 21 of tumor growth (day 14 of treatment) and as can be seen in FIG. 1 the ¹³¹I-VCN show a pronounced lower number of photons observed in the tumor and the body condition of the ¹³¹I alone mouse shows acute distress as compared to the mouse treated with ¹³¹I-VCN. In determining the overall effect of ¹³¹I-VCN therapy, survival of the mice was followed. FIG. 2A shows the distinct survival advantage in the U87 model with mean survival reaching 35 days as compared to 22 days for control and ¹³¹I-VCN alone. In the invasive U251 model the ¹³¹I-VCN treated group survival was again increased by 10 days as compared to control and ¹³¹I alone (FIG. 2B). In summary ¹³¹I-VCN has shown to be an effective agent for enhancing survival in models of glioma.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control.

Other embodiments are set forth within the following claims.

1. A method for suppressing or inhibiting the growth of a cancer cell or tumor cell that expresses one or more integrins on its cell surface, comprising contacting the cell with an effective amount of contortrostatin (CN) and/or vicrostatin (VCN) or an equivalent of each thereof, wherein the CN and/or VCN or the equivalent of each thereof is conjugated to one or more therapeutic radioisotopes.

2. A method for suppressing or inhibiting the growth of a cancer cell or tumor cell that expresses one or more integrins on its cell surface in a subject in need thereof, comprising administering to the subject an effective amount of contortrostatin (CN) and/or vicrostatin (VCN) or an equivalent of each thereof, wherein the CN and/or VCN or the equivalent of each thereof is conjugated to one or more therapeutic radioisotopes.

3. The method of claim 1, wherein the one or more integrins is selected from the group of αvβ3, αvβ5, or α5β1 or equivalents of each thereof.

4. The method of claim 1, wherein the cancer cell or tumor cell is selected from the group of a breast cancer cell, an ovarian cancer cell, a pancreatic cancer cell, a renal cell carcinoma, a glioblastoma cell or metastatic foci of each thereof.

5. The method of claim 1, wherein the VCN is a polypeptide comprising the amino acid sequence of SEQ ID NO.: 3, or an equivalent thereof.

6. The method of claim 1, wherein the CN is a polypeptide comprising the amino acid sequence of SEQ ID NO.: 1, or an equivalent thereof.

7. The method of claim 1, wherein the therapeutic radioisotope is selected from the group of iodine (I)-125, iodine (I)-124, iodine (I)-131, or iodine (I)-123.

8. The method of claim 1, wherein the CN and VCN or the equivalent of each thereof are conjugated to different therapeutic radioisotopes.

9. The method of claim 1, wherein the CN and VCN or the equivalent of each thereof are conjugated to the same therapeutic radioisotope.

10. The method of claim 1, wherein a plurality of CN or VCN or the equivalent of each thereof is contacted with the cell, and each of the CN or VCN or the equivalent of the plurality of each thereof is conjugated to the same therapeutic radioisotope.

11. The method of claim 2, wherein a plurality of CN or VCN or the equivalent of each thereof is administered to the subject, and each of the CN or VCN or the equivalent of the plurality of each thereof is conjugated to the same therapeutic radioisotope.

12. The method of claim 2, wherein only CN or only VCN or the equivalent thereof respectively, is administered to the subject.

13. The method of claim 1, wherein an equivalent comprises a polypeptide having at least 70% sequence identity to the CN or VCN, respectively.

14. The method of claim 1, wherein an equivalent of CN or VCN comprises a polypeptide encoded by a polynucleotide that hybridizes under stringent conditions to a polynucleotide encoding CN or VCN, respectively or its complement.

15. The method of claim 1, wherein the cell is a mammalian cell.

16. The method of claim 2, wherein the subject is a mammal.

17. The method of claim 16, wherein the mammal is a human patient.

18. The method of claim 2, wherein the CN and/or VCN or the equivalent thereof is administered to the subject by intravenous injection near the cancer or tumor and/or injection into the tumor.

19. A pharmaceutical composition comprising an effective amount of contortrostatin (CN) and/or vicrostatin (VCN) or an equivalent of each thereof, wherein the CN and/or VCN or the equivalent of each thereof is conjugated to one or more therapeutic radioisotopes, and a pharmaceutically acceptable carrier.

20. A kit comprising the pharmaceutical composition of claim 19 and instructions for use.

21. A dosage formulation comprising the pharmaceutical composition of claim 19.
